(19) United States
(12) Patent Application Publication
(10) Pub. No.: US 2025/0268521 A1
(43) Pub. Date: Aug. 28, 2025

(54) Detection of sleep-disordered breathing in children

(71) Applicant: Udisense Inc., New York, NY (US)

(72) Inventors: Tor Ivry, Rishon LeZion (IL); Natalie Louise Barnett, Litchfield, CT (US); Amir Eitan, New York City, NY (US); Dr Maristella Lucchini, New York, NY (US); Shalom Elkayam, Rehovot (IL)

(21) Appl. No.: 19/056,837

(22) Filed: Feb. 19, 2025

Related U.S. Application Data
(60) Provisional application No. 63/556,423, filed on Feb. 22, 2024.

(51) Int. Cl. A61B 5/00; A61B 5/08; A61B 5/11; A61B 5/113; A61B 7/00; G06T 7/00; G06T 7/246; G10L 25/66

(52) U.S. Cl. CPC A61B 5/4818; A61B 5/0077; A61B 5/0816; A61B 5/1118; A61B 5/113; A61B 5/4561; A61B 5/4812; A61B 7/003; G06T 7/0012; G06T 7/246; G10L 25/66; A61B 2503/06; G06T 2207/10016; G06T 2207/30004; G06T 2207/30201

(57) ABSTRACT

A method includes receiving a sequence of video images of a child (26) in a bed (24) captured by an image sensor (74), and a stream of audio data captured, simultaneously with the capturing of the sequence of video images, by a microphone (88) placed in proximity to the child (26). The method further includes extracting first features from the video images relating to motion of the child (26), extracting second features from the audio data relating to sounds produced by the child (26), and correlating the first and second features to generate an indication of sleep-disordered breathing by the child (26). Other embodiments are also described.

### CROSS-REFERENCE TO RELATED APPLICATIONS

The present application claims the benefit of U.S. Provisional Application 63/556,423, entitled “Detection of sleep-disordered breathing in children,” filed Feb. 22, 2024, whose disclosure is incorporated herein by reference.

### FIELD OF EMBODIMENTS OF THE INVENTION

The present invention relates generally to sleep monitoring, and particularly to apparatus, systems and methods for detecting sleep-disordered breathing in children.

### BACKGROUND

The term sleep-disordered breathing (SDB) refers to a continuum of respiratory disorders of different severity, ranging from snoring through to obstructive sleep apnea (OSA). It is estimated that 5% of children suffer from SDB, which if undiagnosed and untreated can lead to severe medical complications, including higher risk of cardiometabolic health problems and lower cognitive performance. Currently, however, the process for diagnosis is cumbersome and expensive. It requires an overnight stay at a sleep laboratory and an assessment performed with multiple sensors. Due to the high cost, limited facility spaces, and burden for the families, this screening is often done only for the most severe cases, leaving a high proportion of children without a feasible solution.

Video-based sleep monitors for infants are known in the art. For example, PCT International Publication WO 2017/196695, whose disclosure is incorporated herein by reference, describes a video monitoring system, which includes a camera head, including an infrared illumination source and an image sensor. A mount is configured to hold the camera head in a fixed location and orientation above a crib, so that the image sensor captures images of the crib and an intervention region adjacent to the crib from a fixed perspective.

Respiration monitors for infants based on video sensing are also known in the art. For example, U.S. Pat. No. 10,874,332, whose disclosure is incorporated herein by reference, describes a system for respiration monitoring using a garment, which is fitted snugly around a body of a human subject, and which includes, on at least a portion of the garment that fits around a thorax of the subject, a pattern of light and dark pigments having a high contrast at a near infrared wavelength. A camera head is mounted in proximity to a bed in which the subject is to be placed, and includes an image sensor and an infrared illumination source, which illuminates the bed with radiation at the near infrared wavelength, and transmits a video stream of images of the subject in the bed captured by the image sensor to a processor, which analyzes movement of the pattern in the images in order to detect a respiratory motion of the thorax.

### SUMMARY

Embodiments of the present invention that are described herein provide systems and methods that can be used to screen for and monitor SDB in the home. SDB in this context is defined based on the presence of apnea and hypopnea events. Obstructive sleep apnea occurs when breathing is interrupted during sleep, for example for a period longer than 10 seconds at least five times per hour (on average) throughout the sleep period. Hypopneas occur when breathing is reduced, leading to reduced oxygen intake.

Systems in accordance with embodiments of the invention can be used in first-line diagnosis of children (i.e., infants or older children) who may have SDB, to give their parents and healthcare providers an indication of whether to invest in a sleep lab assessment. Such a system can also be used for monitoring children as they wait to be assessed in a sleep lab, since often there are long waiting lines. This monitoring gives healthcare providers a more comprehensive view of the patient, since sleep lab assessments capture only one night of data. The system will also be used to monitor children after receiving treatment to assess effectiveness, as well as for long-term monitoring of children who are at high risk for SDB, such as children with Down Syndrome or cleft palate.

Embodiments of the present invention provide methods and systems for SDB screening that do not require attaching sensors to the subject of the test. Alternatively, aspects of these embodiments can be applied in conjunction with sensors of other types and may also be used in sleep labs and other professional facilities.

Some of the embodiments that are described herein provide diagnostic methods in which a camera captures a sequence of video images of a child in a bed, while a microphone placed in proximity to the child simultaneously captures acoustic signals and generates a stream of audio data. A processor extracts visual features from the video images relating to motion of the child and extracts acoustic features from the audio data relating to sounds produced by the child. The processor correlates the visual and acoustic features to generate an indication of sleep-disordered breathing in the child.

In the context of the present specification, including the claims, the term “bed” includes, within its scope, any sort of sleeping surface such as a bassinet, a crib, a standard bed (e.g., a toddler bed), a stroller, a standalone mattress, or a floor. Consequently, in the context of the present specification, including the claims, the term “in a bed” includes, within its scope, any sort of sleeping arrangement.

Snoring is a key indicator of SDB, but snoring sounds of small children are often faint and may be difficult to distinguish from background noises in the home. To overcome this difficulty, some embodiments of the present invention apply a machine learning model to distinguish between breathing sounds of the child and background sounds.

The visual features that the processor extracts may include, for example:
The child's respiration rate.
Whether the child is awake or asleep.
Movements of the child's limbs. The processor may apply the detected movements in estimating an activity level of the child.
The child's static pose.
Whether the child's mouth is open or shut.

In the embodiments that are described below, a computer processor combines audio- and video-based features using machine learning and/or other AI techniques to detect apnea and hypopnea events. Collating information from different sources enables the processor to identify different scenarios. For example, central apneas, which are caused by a brief lack of communication between the brain and the muscles that control breathing, typically involve a temporary alteration of the breathing movement but are less likely to be associated with snoring. On the other hand, during obstructive sleep apnea, the chest still moves even if there is no air flow or only partial air flow, and snoring events are likely. The combination of audio and video sensing is thus useful in detecting and distinguishing automatically between different types of apneas and hypopneas.

On the other hand, although alterations of breathing patterns, snoring, body pose and movements can all be related to apnea and hypopnea events, they can also occur in other circumstances. For instance, snoring can occur due to nasal congestion, and alteration in breathing could occur due to coughing. In embodiments of the present invention, audio features such as the frequency content and complexity of the breath sounds are applied in differentiating between actual obstructive sleep apneas and these alternative situations. Additionally or alternatively, the processor may analyze temporal patterns of snoring events, such as irregular snoring, to predict possible sleep apneas.

In some embodiments, the processor analyzes the video data to extract sleep/wake patterns, which give valuable cues for detection of SDB. On this basis, at the most basic level, the processor will determine that an apnea or hypopnea event has occurred only when the child is asleep, ignoring other noises produced by the environment or the child when awake. In addition, apnea events tend to be associated with particular phases of sleep patterns and tend to occur more frequently toward the end of the night. Thus, the processor may extract and apply information on sleep patterns to assess the probability that a certain set of features constitutes an actual apnea or hypopnea event.

Additionally or alternatively, the processor may analyze the video data to extract features of the child's body pose, motion, and facial expression, as keys for assessing the probability that a given set of features is associated with SDB. For example, detection that the child is in a supine position and/or that the child's mouth is open increases the likelihood that a suspected snoring episode is associated with obstructive sleep apnea.

Although each of the video and audio features noted above might be ambiguous in itself, the combination of video and audio features, together with a well-trained machine learning model, as provided by embodiments of the present invention, enables the processor to classify SDB-related events with high confidence.

There is therefore provided, in accordance with some embodiments of the present invention, a method including receiving a sequence of video images of a child in a bed captured by an image sensor, and a stream of audio data captured, simultaneously with the capturing of the sequence of video images, by a microphone placed in proximity to the child. The method further includes extracting first features from the video images relating to motion of the child, extracting second features from the audio data relating to sounds produced by the child, and correlating the first and second features to generate an indication of sleep-disordered breathing by the child.

In some embodiments, extracting the first features includes computing a respiration rate of the child.

In some embodiments, extracting the first features includes ascertaining whether the child is awake or asleep.

In some embodiments, extracting the first features includes detecting movements of limbs of the child.

In some embodiments, extracting the first features includes estimating an activity level of the child responsively to the detected movements.

In some embodiments, extracting the first features includes detecting a static pose of the child.

In some embodiments, extracting the first features includes detecting whether a mouth of the child is open or shut.

In some embodiments, extracting the second features includes detecting snoring sounds.

In some embodiments, detecting the snoring sounds includes distinguishing between breathing sounds of the child and background sounds.

There is further provided, in accordance with some embodiments of the present invention, a system including an image sensor, configured to capture a sequence of video images of a child in a bed, a microphone, configured to capture a stream of audio data, simultaneously with the capturing of the sequence of video images, while placed in proximity to the child, and a processor. The processor is configured to receive the sequence of video images and the stream of audio data, to extract first features from the video images relating to motion of the child, to extract second features from the audio data relating to sounds produced by the child, and to correlate the first and second features to generate an indication of sleep-disordered breathing by the child.

There is further provided, in accordance with some embodiments of the present invention, a computer software product including a tangible non-transitory computer-readable medium in which program instructions are stored. The instructions, when read by a processor, cause the processor to receive a sequence of video images of a child in a bed captured by an image sensor, and a stream of audio data captured, simultaneously with the capturing of the sequence of video images, by a microphone placed in proximity to the child. The instructions further cause the processor to extract first features from the video images relating to motion of the child, to extract second features from the audio data relating to sounds produced by the child, and to correlate the first and second features to generate an indication of sleep-disordered breathing by the child.

The present invention will be more fully understood from the following detailed description of embodiments thereof, taken together with the drawings, in which:

### BRIEF DESCRIPTION OF THE DRAWINGS

FIG. 1 is a schematic isometric and top view showing details of the deployment and use of a monitoring unit over a crib in detection of SDB, in accordance with an embodiment of the invention;

FIG. 2 is a block diagram that schematically shows functional elements of a monitoring unit, in accordance with an embodiment of the invention; and

FIG. 3 is a block diagram that schematically illustrates a method for detecting SDB using audio and video data, in accordance with an embodiment of the invention.

### DETAILED DESCRIPTION

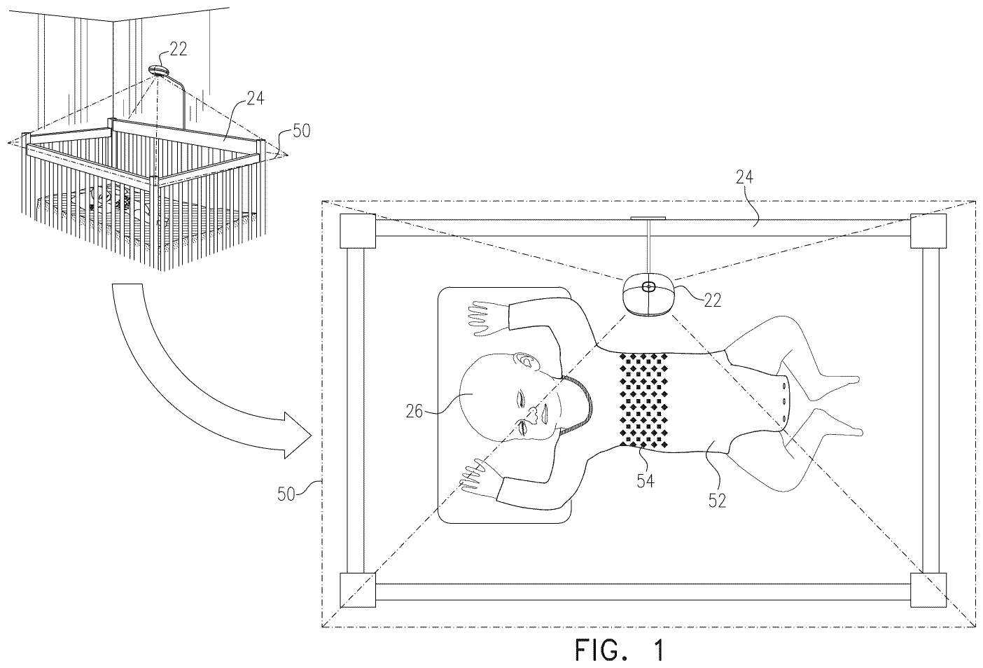

FIG. 1 is a schematic isometric and top view showing details of the deployment and use of a monitoring unit 22 over a crib 24 in detection of SDB, in accordance with an embodiment of the invention. An infant 26 in crib 24 wears a garment 52 with a periodic pattern 54 printed on a portion of the garment that fits around the infant's thorax. Details of this sort of garment and its use in non-contact detection of respiration are described, for example, in the above-mentioned U.S. Pat. No. 10,874,332.

In this embodiment, monitoring unit 22 stands against a wall over crib 24, for example at the midpoint of the long side of the crib. Monitoring unit 22 comprises a camera head having a field of view 50 from a fixed perspective that encompasses at least the area of crib 24. This perspective provides image information that can be analyzed conveniently and reliably to detect respiratory motion, body posture, limb movements, and facial expressions of infant 26. Alternatively, the camera head may be mounted in any other suitable location in proximity to crib 24 that gives a view of the infant suitable for monitoring movement of pattern 54.

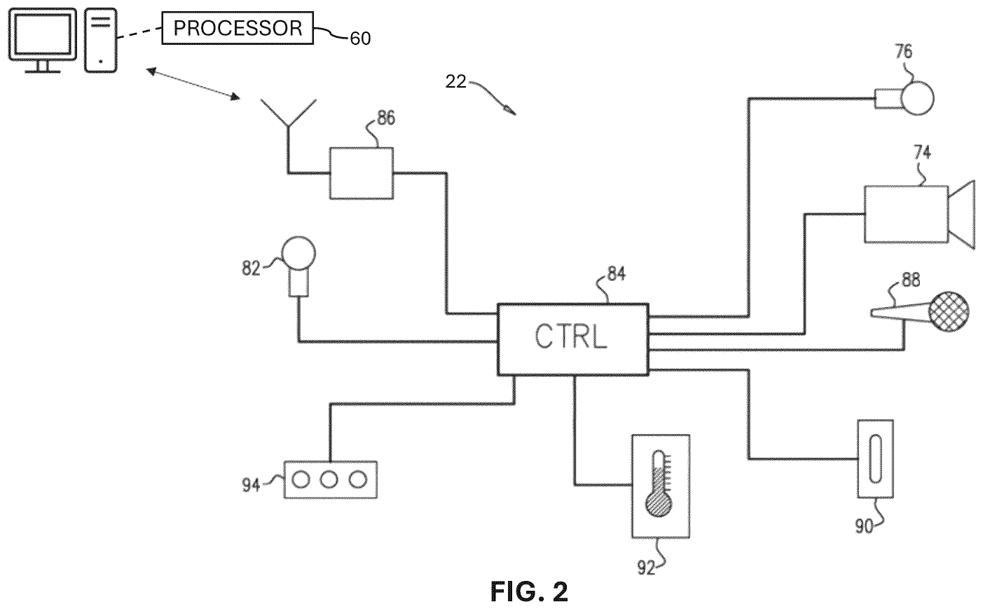

FIG. 2 is a block diagram that schematically shows functional elements of monitoring unit 22, in accordance with an embodiment of the invention. An infrared (IR) light-emitting diode (LED) 76 illuminates the sleeping infant 26. An infrared-sensitive image sensor 74 captures images of field of view 50. A microphone 88 captures audio signals from the area of crib 24. An internal microcontroller (CTRL) 84 coordinates the functions of monitoring unit 22 under control of suitable software or firmware. Microcontroller 84 transmits video and audio data via a communication interface 86 to a processor 60, for example over a wireless network connection.

Processor 60 may be part of a local device, for example in a home computer or smartphone, or it may be in a server, such as a cloud server. The local device or server comprises another communication interface (not shown), via which the processor receives the video and audio data.

Processor 60 may be embodied as a single processor, or as a cooperatively networked or clustered set of processors. The functionality of processor 60 may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, processor 60 may be embodied as a programmed processor comprising, for example, a central processing unit (CPU) and/or a Graphics Processing Unit (GPU). Program instructions, including software programs, and/or data may be loaded for execution and processing by the CPU and/or GPU. The program instructions and/or data may be downloaded to the processor in electronic form, over a network, for example. Alternatively or additionally, the program instructions and/or data may be provided and/or stored on non-transitory tangible media, such as magnetic, optical, or electronic memory. Such program instructions and/or data, when provided to the processor, produce a machine or special-purpose computer, configured to perform the tasks described herein.

Other components and features of monitoring unit 22 are described in the above-mentioned PCT International Publication WO 2017/196695. These components may include, for example, a night light 82, an audio speaker 90, temperature and humidity sensors 92, and status LEDs 94.

Alternatively to an infant, monitoring unit 22 may be used for monitoring an older child, such as a toddler.

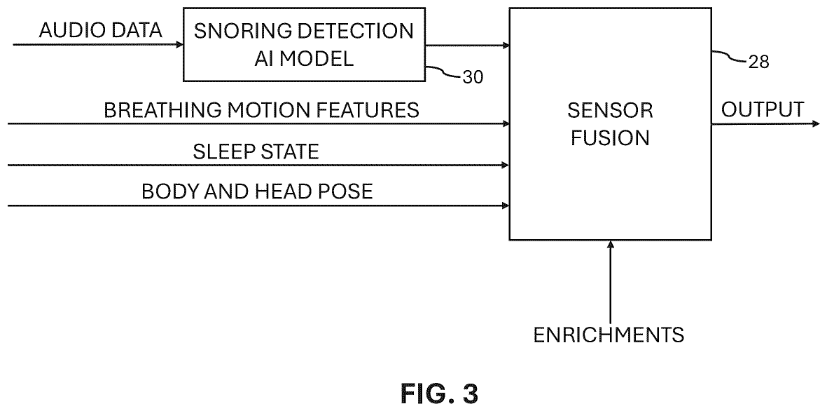

FIG. 3 is a block diagram that schematically illustrates a method for detecting SDB using audio and video data, in accordance with an embodiment of the invention. In the description that follows, it is assumed, for the sake of convenience and clarity, that the method is carried out using the components shown in FIGS. 1 and 2 and described above. Alternatively or additionally, other sources of audio and video data may be used.

Processor 60 extracts audio and video features from the audio and video data collected by monitoring unit 22. The audio and video input features may include (without limitation):
1. Snoring information from the audio.
2. Breathing motion, since apnea and hypopnea events are associated with pauses in breathing movement or shallower breathing movement. In addition, children suffering from SDB exhibit breathing variability even during periods in which apnea and hypopnea events are not occurring.
3. Sleep metrics, particularly metrics indicative of wakeful periods, because SDB is associated with poor sleep efficiency and increased night waking episodes.
4. Open eyes/mouth detection, since children with SDB are more likely to awaken during the night and/or breathe through the mouth.

Other ancillary features may also be collected and input, for example:
5. Demographic information, particularly age and maturity, since preterm babies are more likely to present SDB symptoms.
6. Notes from parents input using a “diaries and logging” feature of the system. These notes may indicate, for example, that the child is sick, which might lead to snoring episodes unrelated to SDB diagnosis.
7. Temperature and humidity information, since dry air is associated with higher chances of snoring events unrelated to SDB.

Processor 60 inputs these features to a sensor fusion model 28, which generates a diagnostic output indicating the times, nature, and severity of episodes of suspected SDB occurring during each monitoring period. Sensor fusion model 28 can use various classification algorithms, for example a support vector machine (SVM), which defines a hyperplane that separates diagnostic classes. The SVM can be a linear or non-linear SVM (using a Radial Basis Function kernel, for example). An advantage of using an SVM in the present embodiment is that the support vectors are determined by the edge samples, so that many events without apnea or hypopnea can be used to improve classification accuracy.

The diagnostic output of the sensor fusion model may include an estimated likelihood that the child suffered from or is likely to suffer from sleep apnea and/or hypopnea and recommendations to seek professional diagnosis. Additionally or alternatively, the output of the sensor fusion model may include other statistics, analysis, and recommendations regarding the child's sleeping patterns.

### Audio-Based Features

For purposes of SDB detection, processor 60 extracts features of audio data captured by microphone 88 and inputs the features to a snoring detection AI model 30. Model 30 is trained on a population of children (e.g., infants), some of whom have exhibited episodes of snoring and some who have not. An audio dataset is collected and annotated by experts to identify periods of snoring and periods of no snoring. This step serves a double purpose: to differentiate between snoring events and non-snoring events and to provide meta-information, such as environmental noises, for use in analysis of the results and training of the AI model. For example, for each audio event, a certain number of epochs before and after the event may be assigned a label of SNORING or NOT SNORING, with the addition of metadata such as voices, dog barking, television, snoring but not from the child, etc. The epochs typically have a duration of about 30 sec, although shorter or longer epochs may alternatively be used.

The resulting database is then used in training the AI model to detect snoring in children. Since microphone 88 in monitoring unit 22 has been used to collect the data, the AI model can be optimized for use with this monitoring unit. The model is likewise optimized for acoustic signal properties, such as spectral features, formant structures, and loudness, of the snoring sounds of children, rather than adults. These signal properties are different, inter alia, due to the differences in upper airway anatomy between children and adults. The AI model may comprise a convolutional neural network, for example. Alternatively, the AI model may be based on any other suitable types of classifiers and machine learning techniques that are known in the art.

The features extracted by processor 60 from the audio data and incorporated in the AI model may include the spectrogram of the audio signals, to leverage both the frequency and a time signature of snoring. The spectrogram is a representation of the spectrum of frequencies of a signal as it varies with time. Processor 60 typically computes spectrograms over short epochs, on the order of 1-10 sec. Each snoring event will thus be covered by multiple spectrograms, thereby increasing the likelihood of correct identification.

### Video-Based Features

Processor 60 processes video images captured by image sensor 74 to extract features and parameters relating to SDB. Although each of the features in itself may not be unique to SDB, the correlation of these features with audio snoring detection gives a reliable indication that infant 26 (or any other child being monitored) is suffering from SDB with some degree of severity. The video-based features that are monitored typically include some or all of the following:

### Respiratory Motion Monitoring:

As described in the above-mentioned U.S. Pat. No. 10,874,332, image sensor 74 captures images of pattern 54 on garment 52, which fits snugly around the thorax of the infant. Pattern 54 is made up of light and dark pigments having a high contrast at a near infrared wavelength and thus can be seen clearly in images captured under infrared illumination, for example illumination by LED 76. Monitoring unit 22 transmits a stream of images to processor 60, which analyzes movement of the pattern in the images in order to detect respiratory motion of the thorax. Based on this analysis, processor 60 outputs respiratory features including the current rate of respiration, as well as the variability of the respiration rate and the amplitude of respiratory motion.

### Body and Head Pose:

Processor 60 analyzes the images output by image sensor 74 to identify locations of joints and other key landmarks in images of the body of infant 26, and uses these points in constructing a geometrical skeleton. (This “skeleton” is a geometrical construct connecting joints and/or other landmarks identified in an image, which does not necessarily correspond to the infant's physiological skeleton.) The locations identified by processor 60 may include the top of the infant's head, the bottom of the infant's neck, the center of the hip, the knees, and the bottoms of the feet, for example. Alternatively or additionally, other points may be identified. Processor 60 may identify these locations automatically, using methods of image processing that are known in the art, or with the assistance of a user.

In addition, the processor can estimate the visibility of the joints. By using the joint visibility, the processor can estimate whether the infant is covered by objects like sleeping bags, or whether the infant's face is covered. For this purpose, a neural network may be trained to extract both joint location and joint visibility, for example by using a set of training images in which parts of the body and limbs are covered in some of the images. Alternatively, the level of joint visibility may be inferred from the variability in joint locations computed by a number of slightly different neural networks, since the detected locations of joints with low visibility are likely to exhibit higher variability from one neural network to another. When visibility is found to be low, the processor may stop the screening and alert the caregiver, thus improving system performance and reliability.

The skeleton that is extracted from the video images is further processed to extract features of the body and head pose, for example:
Is the infant lying down, sitting, or standing?
When the infant is lying down, what is the sleep posture indicated by the main skeleton axis and joints (for example, prone, supine, or lateral)?
How is the head angled relative to the skeleton?

Processor 60 also analyzes changes in the infant's pose and limb positions over a sequence of video frames to extract motion features, indicating the amplitude, speed, and frequency with which the infant moved during sleep. For this purpose, for example, body position information is translated into an activity score. Infants with SDB are known to have more frequent night awakenings, leading to increased movement activity during the night.

If the body and head pose indicate that the infant's face is visible, processor 60 may also detect and analyze the infant's facial features to identify facial expressions, and particularly whether the mouth is open or shut. For example, the processor may analyze the image of the infant's face to track the mouth location and detect changes in the mouth state based on changes in pixel parameters within a bounding box around the mouth location. Additionally or alternatively, a neural network may be trained using images of infant faces that have been annotated to indicate whether the mouth is open or closed.

### Sleep/Wake Patterns:

Processor 60 analyzes the motion features described above to identify patterns of motion of the infant in the crib, giving an output that is similar to actigraphic monitoring. These actigraphic patterns are translated into sleep/wake states as a function of time over the course of the night. Methods for detecting sleep activity, including periods of waking, that can be used for these purposes are described, for example, in the above-mentioned PCT International Publication WO 2017/196695, as well in U.S. Pat. No. 9,530,080, whose disclosure is also incorporated herein by reference.

In addition to the audio- and video-based features described above, the analysis performed by processor 60 may be enriched with other data, such as the outputs of other sensors in or associated with monitoring unit 22.

The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method, comprising:
receiving:
a sequence of video images of a child in a bed captured by an image sensor, and
a stream of audio data captured, simultaneously with the capturing of the sequence of video images, by a microphone placed in proximity to the child;
extracting first features from the video images relating to motion of the child;
extracting second features from the audio data relating to sounds produced by the child; and
correlating the first and second features to generate an indication of sleep-disordered breathing by the child.

2. The method according to claim 1, wherein extracting the first features comprises computing a respiration rate of the child.

3. The method according to claim 1, wherein extracting the first features comprises ascertaining whether the child is awake or asleep.

4. The method according to claim 1, wherein extracting the first features comprises detecting movements of limbs of the child.

5. The method according to claim 4, wherein extracting the first features comprises estimating an activity level of the child responsively to the detected movements.

6. The method according to claim 1, wherein extracting the first features comprises detecting a static pose of the child.

7. The method according to claim 1, wherein extracting the first features comprises detecting whether a mouth of the child is open or shut.

8. The method according to claim 1, wherein extracting the second features comprises detecting snoring sounds.

9. The method according to claim 8, wherein detecting the snoring sounds comprises distinguishing between breathing sounds of the child and background sounds.

10. A system, comprising:
an image sensor, configured to capture a sequence of video images of a child in a bed;
a microphone, configured to capture a stream of audio data, simultaneously with the capturing of the sequence of video images, while placed in proximity to the child; and
a processor, configured to:
receive the sequence of video images and the stream of audio data,
extract first features from the video images relating to motion of the child, extract second features from the audio data relating to sounds produced by the child, and
correlate the first and second features to generate an indication of sleep-disordered breathing by the child.

11. The system according to claim 10, wherein the processor is configured to extract the first features by computing a respiration rate of the child.

12. The system according to claim 10, wherein the processor is configured to extract the first features by ascertaining whether the child is awake or asleep.

13. The system according to claim 10, wherein the processor is configured to extract the first features by detecting movements of limbs of the child.

14. The system according to claim 13, wherein the processor is configured to extract the first features by estimating an activity level of the child responsively to the detected movements.

15. The system according to claim 10, wherein the processor is configured to extract the first features by detecting a static pose of the child.

16. The system according to claim 10, wherein the processor is configured to extract the first features by detecting whether a mouth of the child is open or shut.

17. The system according to claim 10, wherein the processor is configured to extract the second features by detecting snoring sounds.

18. The system according to claim 17, wherein detecting the snoring sounds includes distinguishing between breathing sounds of the child and background sounds.

19. A computer software product comprising a tangible non-transitory computer-readable medium in which program instructions are stored, which instructions, when read by a processor, cause the processor to:
receive:
a sequence of video images of a child in a bed captured by an image sensor, and
a stream of audio data captured, simultaneously with the capturing of the sequence of video images, by a microphone placed in proximity to the child, extract first features from the video images relating to motion of the child,
extract second features from the audio data relating to sounds produced by the child, and
correlate the first and second features to generate an indication of sleep-disordered breathing by the child.

20. The computer software product according to claim 19, wherein the instructions cause the processor to extract the first features by detecting movements of limbs of the child.
